(19) United States
(12) Patent Application Publication
(10) Pub. No.: US 2009/0276864 A1
(43) Pub. Date: Nov. 5, 2009

(54) TRANSGENIC ANIMALS WITH REDUCED MAJOR URINARY PROTEIN PRODUCTION

(76) Inventors: Martin John Evans, Cardiff (GB); Fiona Catherine Mansergh, Cardiff (GB); Jane Louise Hurst, S Wirral (GB); Robert Jeffrey Beynon, Liverpool (GB)

Correspondence Address: KOHN & ASSOCIATES, PLLC, 30500 NORTHWESTERN HWY, SUITE 410, FARMINGTON HILLS, MI, 48334, US

(21) Appl. No.: 11/720,730

(22) PCT Filed: Dec. 5, 2005
(86) PCT No.: PCT/GB05/04674

§ 371 Date: Jun. 18, 2009

(30) Foreign Application Priority Data
Dec. 3, 2004 (GB) 0426650.8

(51) Int. Cl. G01N 33/48; A01K 67/027; C12N 15/85; C12N 15/63; C12N 5/00

(52) U.S. Cl. 800/3; 800/14; 800/18; 800/21; 435/320.1; 435/325

(57) ABSTRACT

The present invention relates to a transgenic animal having substantially reduced major urinary protein (uMUP) production. The present invention also relates to methods of producing such animals, and to the uses of such animals.

The present invention relates to a transgenic animal having substantially reduced major urinary protein (uMUP) production. The present invention also relates to methods of producing such animals, and to the uses of such animals.

Many animals rely on scent communication to transmit information between individuals about, for example, sex, age, development, identity, reproductive state, and social and health status. Some information is transmitted during contact between individuals, but complex information can also be ascertained from urine scent marks deposited in the environment. The inventors were concerned with understanding how this complicated information can be conveyed in the urine, and how it can be linked to the depositing animal, and also with the outcome for animals if they fail to express these proteins.

Previous research has concentrated on the volatile components of scents. Involatile proteins termed major urinary proteins (uMUPs) have been revealed to be important. These proteins are lipocalins which are synthesised in the liver and eliminated in urine. The proteins bind and release pheromones, and the unique pattern of uMUPs expressed by an individual acts as a code to define the identity of that individual.

Mouse urine contains a high concentration of proteins termed major urinary proteins (uMUPs), whose only known functions are in chemical signalling. These small proteins bind a wide-range of male-specific volatile pheromones (Bacchini et al. 1992; Robertson et al 1993) and elicit a slow release of these volatiles from the urine scent marks that males deposit throughout their territory (Hurst et al. 1998). These male-specific volatiles are used in competitive communication between males, stimulating aggression (Novotny et al. 1985), attraction (Humphries et al. 1999; Mucignat-Caretta & Caretta 1999) or aversion (Novotny et al. 1985) depending on the context and social status of scent owner and receiver. They are also highly attractive to females (Jemiolo et al. 1985) and have a number of priming effects on female reproductive physiology (Jemiolo et al. 1985, 1986; Novotny et al. 1999). uMUPs thus influence the chemical profile and release of airborne signals, greatly extending the duration over which territorial signals attract the attention of conspecifics (Hurst et al. 2001a).

Involatile uMUPs and their bound ligands also play a more direct role in signalling. Scent marks deposited in the environment can only provide information about the owner if the scent contains a stable and persistent signal of owner identity. Genetically heterogeneous wild mice express many uMUP polymorphic variants, each individual expressing around 7 to 14 variants (Payne et al. 2001). The combinatorial diversity of uMUPs is thus extremely high (potentially as great as for MHC). The inventors have shown that uMUPs provide genetically-determined individuality signatures in the urine scent marks of male mice (Hurst et al. 2001b). While many genetic and non-genetic factors influence the volatiles in mouse scent marks that stimulate mice to contact the scent to gain more information, it is the pattern of uMUPs in the scent mark that provides the ownership signature (reviewed by Hurst et al. 2004). Since contact investigation is necessary to detect scent ownership, the individuality signature must be provided by the involatile uMUPs or uMUP-ligand complexes rather than by airborne volatiles released by uMUPs (Nevison et al. 2003).

It is widely assumed that uMUPs are a male-specific trait, involved in male competitive and sexual signalling. This is based on early studies of laboratory mice which suggested that uMUP levels were 20-30 times higher among males (e.g. Szoka & Paigen 1978). However, the inventors' surveys of wild-caught and wild-derived mice, together with a large range of laboratory strains, indicate that males produce only 3-4 times as much on average as females, consistent with the much higher rate of scent marking required by male mice to advertise territory ownership (Hurst 1993). uMUP production is still substantial among female mice (7.5±0.9 mg/ml urine among n=18 wild-caught females), which have a considerably greater output than males in many other rodent species (e.g. Kruczek & Marchlewska-Koj 1985; Leeman-McKeeman & Caudill 1991; Cain et al. 1992; unpublished data). The pattern of uMUPs expressed differs between the sexes (Finlayson et al. 1963; Payne 2002). This may be due to androgen-specific expression of some uMUPs (Finlayson et al. 1963), although the complexity of uMUP patterns expressed by wild-caught or wild-derived females is no less than that of males (Payne et al. 2001). Nothing is known about the function of MUPs in female urine, but it is likely that uMUPs play a much wider role in sex and identity recognition and that these involatile proteins are much more important in the detection and recognition of social signals than has previously been supposed.

In strong support of this, recent studies suggest that the mouse accessory olfactory system normally detects only involatile social signals. Although V1R receptors in the vomeronasal organ (VNO) respond to volatile pheromones in VNO slices (Dulac & Axel 1995; Leinders-Zufall et al. 2000), electrophysiological recordings in freely behaving laboratory mice investigating an anaesthetised conspecific reveal that neurones in the accessory olfactory bulb (which receives input from the VNO) are activated only when the nose makes direct contact with the source of social odours (Luo et al. 2003). Low molecular weight pheromones presented alone are ineffective and appear not to be detected through the vomeronasal system but are detected when combined with urinary proteins (Yamaguchi et al. 2000). This is consistent with the idea that the VNO primarily accesses odorants bound to involatile proteins such as uMUPs through an active pumping mechanism (reviewed by Halpern & Marcos 2003). Individual Anterior Olfactory Bulb (AOB) neurones respond selectively depending on the specific combination of sex and strain of stimulus animals (Luo et al. 2003) while recognition of sex is impaired in genetic mutants with non-functional VNO receptors (Dulac & Torello 2003). The involatile scent components delivered to the VNO thus appear to provide information about the sex and identity of the scent owner.

uMUPs and their bound ligands are the most likely candidates for delivering social identity signals to VNO receptors, although no one has yet been able to examine their role during direct interactions between conspecifics rather than isolated scents. MUPs have evolved to bind volatile pheromones, are by far the most prevalent involatile components of mouse urine, comprising more than 99% of urinary proteins (Humphries et al. 1999), exhibit sufficient complexity and stability (Beynon et al., 2001) and have already been shown to provide the ownership signal in male scent marks. It is not yet known whether information is provided only by low molecular weight ligands delivered to receptors by uMUPs or whether the uMUPs themselves also convey information, as uMUP receptors have not been definitively identified (Dulac & Torello 2003; Beynon & Hurst. 2004).

Social information might only be gained through direct contact with involatile components such as uMUP-ligand complexes. However, there is abundant evidence that mice recognise familiar and unfamiliar volatile social stimuli without contacting the scent source. When mice and rats encounter a complex of highly familiar volatiles, they do not contact the scent source to investigate further (e.g. Singh et al. 1987; Hurst 1993). But when volatiles are less familiar or have not been encountered recently (or at that location), this stimulates prolonged close investigation. When in contact with the scent source, involatile uMUP-ligand complexes are detected through the VNO while volatiles in the scent simultaneously stimulate activity in the main olfactory bulb through the main olfactory epithelium (Guo et al. 1997). This provides animals with the opportunity to learn an association between social signals, for which there are specific receptors in the VNO, and the complex mixture of volatiles in scents that can be detected through the main olfactory epithelium but which are influenced by many genetic and environmental factors such as food type, bacterial flora, social status and infection (Brown 1995; Penn & Potts 1998a).

Learning this association may be essential to allow mice to recognise both the sex and identity of a conspecific from their volatile scents alone. Female mice that have never previously encountered the scents of adult males show an innate attraction to male scents they are able to contact. By contrast, naïve females show no such innate attraction if contact is prevented and they detect only airborne volatiles (Moncho-Bogani et al. 2002). However, if repeatedly allowed full contact with adult male scents, females learn an association between the involatile and volatile components and, subsequently, are attracted to the volatile components of male scents alone. A similar mechanism may apply to male recognition of females. Male mice emit ultrasonic (70 kHz) courtship vocalizations specifically in response to females or their scents. However, males that have never physically contacted a female or female scent after weaning fail to emit vocalisations to female urine, suggesting that they too must learn the signal value of female urine as a result either of physical experience with a female or with her scent (Dizinno et al. 1978). The components of female urine that elicit courtship vocalizations are non-volatile and environmentally stable, stimulating strong responses even 7 days after deposition and remain active for at least a month (Nyby & Zakeski 1980). However, after encountering females artificially odorized with perfume, male mice will subsequently emit courtship vocalizations to the perfume alone (Nyby et al. 1978). Similar mechanisms may also apply to individual recognition or to the recognition of kin. While highly familiar members of a social group are recognised without contact and induce no further investigation, unfamiliar animals induce prolonged contact investigation, particularly around the anogenital region. Numerous experiments have demonstrated that mice imprint on volatile odorants that have been applied or fed to their parents during rearing, subsequently influencing their mate preference (eg D'Udine & Alleva 1983).

Many genes influence the volatile scents produced by animals, especially those of the highly polymorphic MHC (e.g. mice: Yamazaki et al. 1979; rats: Singh et al. 1987; humans: Wedekind et al. 1997, Jacob et al. 2002). Laboratory studies utilizing MHC congenic strains have confirmed that both mice and rats are able to discriminate airborne urinary odours when donors differ genetically only at alleles within the MHC region (Singh et al. 1987; Carroll et al. 2002). These odours promote MHC disassortative mating (Potts et al. 1991) and kin recognition (Manning et al. 1992) between interacting mice. However, MHC is neither sufficient nor necessary for the recognition of individual scent mark owners. Mice learn to recognise the volatile odours of familiar neighbour competitors resulting from both MHC and genetic background, which induces prolonged investigation of their scent marks; but once closely investigated MHC-associated odours induce no further response (Hurst et al., to be published 2005). This is in contrast to uMUP-associated scents, which on close investigation allow recognition and countermarking of scents from specific individual neighbours.

The molecular basis of MHC-associated odours is not known, but appears to involve a complex mixture of volatile metabolites bound and released by urinary proteins (Singer et al. 1993, 1997). It is not known whether the urinary proteins involved are fragments of MHC molecules themselves, or are uMUPs. The “carrier hypothesis” (Singh 2001) proposes that soluble fragments of MHC class I and class II molecules in urine differentially bind volatile metabolites in the antigen-binding groove once the peptide that is normally bound tightly in this groove is lost during the fragmentation process. Further proteolysis of the fragments would then lead to release of the volatiles. The MHC specificity of odours is thus determined by the highly polymorphic binding characteristics of the antigen-binding groove. How low molecular weight volatiles could be specifically bound to MHC protein fragments that normally bind peptides is unclear though (Singer et al. 1997). By contrast, the central calyx of uMUPs is designed to bind small odorant molecules and uMUPs are present in considerably greater concentration than MHC fragments in urine (Beynon et al. 2001). It is also well established that many physiological traits are genetically associated with the MHC and are likely to influence metabolites (e.g. Ivanyi 1978). Thus, an alternative hypothesis for the molecular basis of MHC-associated volatiles is that MHC-based developmental and physiological variations give rise to distinct volatile profiles .(Boyse et al. 1987) which are then bound and released by uMUPs (Beynon & Hurst 2004).

These two mechanisms have different implications for the potential importance of MHC-associated odours in communication. If the carrier hypothesis is correct, there is a specific and separate mechanism for chemical communication of MHC type in which involatile MHC fragments may deliver MHC-specific information to the VNO independently of information carried by uMUPs. If the uMUP hypothesis is correct, then volatile ligands reflect general physiological traits that are influenced by many genetic and environmental factors and will provide much less specific information about MHC type. It would also mean that two highly polymorphic multigene complexes inherited independently on separate chromosomes act together to determine the main genetic basis of an individual's volatile and involatile urinary profile. The capacity for unique combinations of these components is considerable. Discriminating between these two mechanisms is thus extremely important to understand the molecular, functional and evolutionary significance of MHC-associated odours. However, biochemically it is not feasible to separate uMUPs and MHC fragments completely for use in bioassays of recognition, and any attempt at manipulation of urine components in vitro cannot be applied in functional tests such as mate choice that require direct interaction between animals producing scents.

In order to establish whether uMUPS and their ligands are essential involatile components for the recognition of characteristics about the depositing animal, and whether uMUPs are responsible for the binding and release of MHC-associated odours, it would be useful to be able to manipulate the presence and absence of uMUPs and their bound ligands at source.

Further, it would be advantageous to provide an animal having substantially reduced uMUP production, for example for use in laboratories, animal houses, animal breeding centres, etc. A large number of people working with animals, particularly in such establishments, develop an animal allergy. Some go on to develop asthma. It would be advantageous to provide animals which are hypoallergenic. One of the main causes of animal allergy, particularly in laboratories and animal houses, etc., is lipocalins. By reducing the production of uMUPs, which are lipocalins, the induction of allergies will also be greatly reduced.

Shahan et al., (1987) describes the expression of six mouse uMUPs in different tissues and indicates that mouse uMUPs are encoded by a family of about 35 to 40 conserved genes.

Held et al., (1987) describes the identification of genes encoding mouse uMUPs.

Utsumi et al., (1999) describes the expression of mouse uMUPs in nasal tissue. It is also indicated that the mouse genome contains about 35 uMUP genes, many of which are clustered on chromosome 4.

Johnson et al., (1995) describes the sexual dimorphism and mechanisms of uMUP production. It is also indicated that members of a subgroup of 15 to 20 uMUP genes are located in a cluster on chromosome 4.

Held et al., (1987) and Johnson et al., (1995) also make reference to the lit/lit mouse which is a growth hormone deficient strain. The lit/lit mouse is a dwarfed mouse, and, along with a number of other abnormalities, shows approximately 1% of normal adult uMUP levels.

Duncan et al., (1988) describes a survey of uMUPs in laboratory mice, in particular in the substrain BALB/cJPt. BALB/cJPt has two uMUP phenotypes, one of which, designated the “null phenotype” produces less uMUPS than the other. This mouse is not a true “null-MUP” because it does still produce a substantial amount of uMUPs. There is no suggestion in the prior art to knock out the uMUP genes due to the understanding that it was not feasible to produce a transgenic animal with reduced or eliminated uMUP production since there are more than 20 distinct genetic sites which have homology to uMUPs and may therefore encode one or more uMUPs. Those skilled in the art did not consider producing a mouse without uMUP production due to the large number of genes that would need to be knocked out as well as expected difficulties with breeding mice with no uMUP production. Surprisingly, the inventors found that it is possible to produce a transgenic animal which shows substantially reduced, if not eliminated, uMUP production.

According to the invention, there is provided a transgenic animal from whose genome a uMUP complex has been inactivated or deleted, wherein said inactivation or deletion substantially reduces the production or secretion of uMUPs.

The term uMUP is used herein to mean the major urinary proteins found in mouse urine and the equivalent proteins found in the urine of other animals, such as, but not limited to the a2u globulins found in rat urine.

The transgenic animal according to the invention may be any animal, preferably a non-human animal, more preferably a non-human mammal, more preferably a rodent, especially a mouse or a rat. Mice are especially preferred. Any mouse strain may be used to produce the transgenic animal of the present invention, including in-bred, out-bred, hybrid, mutant (natural mutation), transgenic and congenic strains and wild-derived mice. Preferred strains include 129Sv/Ev and C57B16/J.

The term “wild-derived animal” includes animals captured from the wild or animals bred in captivity for one or more generations from animals captured from the wild.

The term “substantial reduction of uMUP production” means that the transgenic animal has less uMUPs in its urine than is found in wild-type animal urine. The wild-type animal is of the same species, gender and age as the transgenic animal, and is preferably of the same strain as the transgenic animal. The term preferably means the transgenic animal's urine contains less than 10% of the level of uMUPs found in the urine of a wild-type animal, preferably less than 5%, more preferably less than 1%, more preferably less than 0.5%, more preferably less than 0.1%, most preferably less than 0.01%. Most preferably uMUP production is reduced to such a degree that uMUPs cannot be detected in non-concentrated urine by standard techniques such as isoelectric focussing on acrylamide gels immunoassay or mass spectrometry.

Wild-type animal means a common example of an animal that exhibits similar characteristics to those found naturally. In particular it means a non-mutant and non-transgenic animal. The term may apply to wild animals or laboratory strains. It is a term that is clear to those skilled in the art.

When the transgenic animal is a mouse, the wild-type mouse is preferably considered to be a mouse strain derived from (1) C57-related strains (e.g. C57BL/6, C57BL/10, C57BR, C58, C57BLKS and related strains); (2) Castle's mice (e.g. DBA, CBA, C3H, BALB, 129-related strains, A, AKR, NZ and related strains); (3) Swiss mice (e.g. ICR, SWR, SWJ, SJL, FVB, NIH, NMRI, NOD, NOR, HLS and related strains); (4) strains derived from colonies from China and Japan (e.g. DDN, DDP, DM, DRC, KK, KR, RR, KYF, IVCS, FM and related strains); (5) strains derived from wild mice (e.g. SPRET, CAST, CASA, PANCEVO, MOL-related strains); (6) other inbred strains (e.g. RIII, RBC, CPB); (7) outbred strains (e.g. ICR, CD-1, MF1, CF-1, Swiss, CFW, SKH1, PGP, BK) or (8) hybrids (see Beck et al. 2000 for detailed genealogy of inbred strains). When the transgenic animal is a rat, the wild-type rat is preferably considered to be a rat strain derived from (1) an inbred strain (e.g. PVG, Lewis, Fischer, Nude, Brown Norway, SHR, W KY, Noble, Copenhagen, BDIX, Buffalo and related strains) or (2) an outbred strain (e.g. CD, Wistar, Sprague Dawley, Long Evans, Zucker). The concentration of uMUPs in the urine of mice is usually between 1 mg/ml and 50 mg/ml for males and between 0.2 mg/ml and 20 mg/ml for females when uMUP concentration is measured by any protein assay.

Accordingly, a male transgenic mouse according to the invention preferably has a urine uMUP concentration of less than 1 mg/ml, more preferably less than 0.5 mg/ml, more preferably less than 0.1 mg/ml, even more preferably less than 0.05 mg/ml, most preferably less than 0.01 mg/ml.

Accordingly, a female transgenic mouse according to the invention preferably has a urine uMUP concentration of less than 0.2 mg/ml, more preferably less than 0.1 mg/ml, more preferably less than 0.05 mg/ml, even more preferably less than 0.025 mg/ml, most preferably less than 0.01 mg/ml.

Urine from male uMUP-null mice defined in Duncan et al. (1988) contained only four of the eight uMUP bands seen in normal wild-type BALB/cAn males, while urine from female uMUP-null mice contained between three and eight of the eight uMUP bands seen in normal wild-type BALB/cAn females. The relative quantity of uMUP excreted by these uMUP-null mice was significantly less than the mean uMUP concentration of urine from normal wild-type BALB/cAn mice but varied between individuals. The relative uMUP concentration of male uMUP-null mice was approximately 35% of mean values for normal wild-type BALB/cAn males (range 23 53%) while uMUP concentration of female uMUP-null mice was approximately 19% of mean values for normal wild-type BALB/cAn females (range 12 22%).

It is preferred that the transgenic animal is phenotypically identical to a wild-type animal except for the reduction or absence of uMUP production. In particular, it is preferred that the transgenic animal has no physiological or morphological abnormalities. By ensuring that the transgenic animal has no physiological or morphological abnormalities it means that the animal can be used in research.

It has been found that substantially all uMUPs are encoded by a single gene cluster. This is known herein as the uMUP complex. In mice this complex is found on chromosome 4. By inactivating, preferably by deleting, this single complex uMUP production is substantially reduced. Similar complexes are found in other animals, in particular, the rat, in which the equivalent proteins are termed a2u globulins encoded by genes on chromosome 5.

Inactivation of the uMUP complex is taken to mean preventing the expression of functional uMUPs from the complex. This can be done, for example, by deleting the complex or by replacing it with a non-functional complex, or by mutating the complex so that expression of functional uMUPs is prevented. Preferably the uMUP complex is inactivated by deleting the entire complex. It is preferable to delete the complex so that there is no chance of an inactivated complex reverting to an active form, from which the expression of functional uMUPs is possible.

A functional uMUP is a uMUP that is able to bind pheromones and/or other ligands involved in the transmission of information between animals by scent and is allergenic. An inactivated uMUP does not bind pheromones and/or other ligands and/or is not allergenic. Preferably the inactivated uMUP has neither function.

The complex can be removed by any known method. For example it can be removed by using a single targeting vector with homology arms which embrace the complete uMUP complex. Alternatively, two vectors may be used which flank the uMUP complex, wherein when the two vectors combine, the uMUP complex is deleted.

The vector or vectors may contain a marker, so that cells which have been successfully targeted by the vector may be identified and selected.

Alternatively the uMUP complex can be mutated to prevent expression of functional uMUPs. Methods for mutating the uMUP complex will be apparent to those skilled in the art.

In mice, the uMUP complex is approximately 1 Mbp in size. The complex is found between nucleotides 59224905 and 60414792 bp. The first uMUP gene is MUP4 at 59224905 and 59228764. The last locus in the region having MUP homology is found at 60410982 to 60414792 bp. (This numbering is taken from the Ensembl database as of 15 Nov. 2004). This numbering may change as the nucleotide sequence is refined, but one skilled in the art would be able to locate the loci mentioned.

Distances and memory systems vary according to the database. Examples and comparison between databases are provided in the detailed description. One skilled in the art would be able to work out the position of the nucleotides discussed herein, and to work out the location of the various elements.

The uMUP complex is flanked by regions which can be used by targeting vectors for removal of the complex. In particular, a Tscot region is found between nucleotides 59173225 and 59183155. A zinc finger gene Zfp37 is found between 60517046 and 60535864 bp. (All taken from the Ensembl database as of 15 Nov. 2004.)

The numbering of the nucleotides given above may change as the nucleotide sequence is refined. This would be clear to one skilled in the art. The uMUP complex is preferably found between the 3′ end of the Tscot region and the 5′ end of the Zfp37 gene. The sequence of the Tscot region is provided in SEQ ID No. 1. The sequence of the Zfp37 gene is provided in SEQ ID No. 2.

In rats, the equivalent complex is in the region of 78.4 Mbp on chromosome 5. A Tscot region is found at 78.05 Mbp. A zinc finger gene Zfp37 is found at 79.0 Mbp.

The present invention also provides a method of producing a transgenic animal which has substantially reduced uMUP production, comprising these steps of:
A) Providing embryonic stem cells comprising an intact uMUP complex;
B) Providing one or more targeting vectors capable of inactivating the uMUP complex,
C) Introducing the one or more vectors into the embryonic stem cells and selecting for cells with an inactivated uMUP complex;
D) Introducing an embryonic stem cell having an inactivated uMUP complex into a blastocyst; and
E) Implanting the blastocyst into the uterus of a female animal and allowing it to gestate before delivering the resulting offspring.

The transgenic animal produced by this method may be heterozygous or homozygous for the inactivated uMUP complex.

Preferably the method further includes the step of breeding the resulting offspring with similar offspring in order to produce stable animals which are homozygous or heterozygous for the inactivated uMUP complex.

The one or more vectors used in the above methods may inactive the uMUP complex by any known method. Preferably the vectors delete the uMUP complex. The one or more vectors may be any vectors which target the uMUP complex, or areas which flank it.

If the uMUP complex is to be deleted, the one or more vectors preferably comprise a single targeting vector with homology arms, which embrace the complete uMUP complex. Alternatively, two vectors which flank the uMUP complex may be used. The vectors flanking the complex may preferably comprise LoxP, and bring about Cre-mediated recombination to eliminate the uMUP complex.

The vectors preferably target the Tscot and Zfp37 regions which flank the uMUP complex. Alternatively, the vectors preferably target the regions between the Tscot and Zfp37 regions and the uMUP complex.

Any other type of vector known to one skilled in the art that will inactivate the region may be used. For example vectors which insert a sequence or which bring about mutations throughout the entire region, etc.

The method of producing a transgenic animal according to the invention may further comprise the step of inactivating any residual uMUP sites which are not part of the uMUP complex.

The invention further provides vectors for use in the method of the invention. In particular, the method provides a single targeting vector, with homology arms which embrace the complete uMUP complex. Preferably the vector is pMupsTV1 as described in the example section below.

Alternatively, two vectors which flank the uMUP complex are provided. The vectors flanking the complex preferably comprise LoxP, and bring about Cre-mediated recombination to eliminate the uMUP complex. The vectors include vectors known to one skilled in the art which may be used to inactivate, especially delete, the uMUP complex.

Preferably the vectors include a marker to enable cells successfully targeted with a vector to be identified and selected. When two vectors are provided, the vectors preferably include part of a gene, such as the HPRT gene. When a cell is successfully targeted with the vectors the HPRT gene is reassembled and may be identified. As a result the cells may be selected for in culture.

As an example, details of suitable vectors are given in the example section below.

The present invention also provides animals created by the methods of the invention. For example the invention provides animals which are heterozygous for an inactivated uMUP complex, animals which are homozygous for an inactivated uMUP complex, and all generations of animals which have been bred from animals created by the methods of the invention, or from animals created by the methods of the invention which have been crossed with wild-type animals or wild-derived animals, for example, F₁, F₂, F₃, and F₄ generations. Such animals include animals bred by crossing an animal created by the methods of the invention with another animal created by the methods of the invention. Such animals also include animals bred by crossing an animal created by the methods of the invention with a wild-type animal, and further generations therefrom.

Especially preferred are animals which have been bred from an animal that is homozygous for an inactivated uMUP complex. In particular, animals which have been bred from a dam that is homozygous for an inactivated uMUP complex are preferred. Such animals have not been exposed to a maternal uMUP complex.

Also preferred are animals that have been bred from an animal that is heterozygous for an inactivated uMUP complex.

The present invention also provides an embryonic stem cell that is heterozygous or homozygous for an inactivated uMUP complex. Methods for producing such embryonic stem cells from the transgenic animals of the present invention are well known to those skilled in the art. Preferably the embryonic stem cell is a non-human stem cell, more preferably a rodent (e.g. mouse or rat) embryonic stem cell.

Also provided is a method for selecting animals produced by the methods of the invention comprising testing the animals for the inactivation of the uMUP complex. Testing methods preferably include the step of analysing a sample from a transgenic animal, in particular a urine, tissue or blood sample. The sample may be tested for uMUP concentration, for RNA produced in the expression of uMUPS, or for the presence of an active or inactivated uMUP complex in the animal's DNA. Such testing may be carried out by any method known to one skilled in the art, but particularly by methods such as southern blot analysis of DNA and protein chemistry analysis of urine, such as immunochemical, mass spectrometry and photometric analysis. Tests may also include testing urine for ligand release rates. Ligands normally bound by uMUPs will be released from the urine of a transgenic animal having substantially reduced uMUP production faster than from wild-type animal urine.

In another aspect of the invention, there is provided the use of a transgenic animal having substantially reduced uMUP production in behavioural studies. Preferably the transgenic animal used in such studies is homozygous for the inactivated uMUP complex, but a heterozygous animal may also be used. The transgenic animal used in the behavioural studies may also be an animal bred from an animal having an inactivated uMUP complex. In particular an animal bred from a dam which is homozygous for the inactivated uMUP complex may be used in behavioural studies.

Behavioural studies include determining the role of uMUPs in chemical communication and assessment of the effects of removal of this line of communication. In particular, behavioural studies include, but are not limited to, studies of growth performance, reproductive success, sexual and parental behaviour such as mother:pup recognition, male aggression and same sex aggression. Behavioural studies also include investigation into the recognition of male scent by females and vice versa, and also the recognition of particular individuals.

Also provided by the invention is the use of a transgenic animal according to the invention to establish the molecular mechanism underlying MHC associated odours. Preferably the transgenic animal used in establishing the molecular mechanism underlying MHC associated odours is homozygous for the inactivated uMUP complex, but a heterozygous animal may also be used. The transgenic animal used in establishing the molecular mechanism underlying MHC associated odours may also be an animal bred from an animal having an inactivated uMUP complex. In particular an animal bred from a dam which is homozygous for the inactivated uMUP complex may be used in establishing the molecular mechanism underlying MIC associated odours.

A further aspect of the invention provides the use of transgenic animals according to the invention to identify agents which affect animal behaviour. Preferably the transgenic animal used in identifying agents which affect animal behaviour is homozygous for the inactivated uMUP complex, but a heterozygous animal may also be used. The transgenic animal used in identifying agents which affect animal behaviour may also be an animal bred from an animal having an inactivated uMUP complex. In particular an animal bred from a dam which is homozygous for the inactivated uMUP complex may be used in identifying agents which affect animal behaviour.

Such agents may be agents which, for example, attract or deter animals or which stimulate or reduce aggression. The agents may be, for example, organic compounds, proteins or inorganic compounds.

Transgenic animals produced according to the invention which are homozygous for an inactivated uMUP complex are hypoallergenic. The use of such animals is also provided by the present invention.

Hypoallergenic means that the animal produces much fewer allergens, which cause an allergic reaction, than wild-type animals.

The availability of a hypoallergenic animal for use in any activity wherein a human comes into contact with the animal, or with a uMUP produced by the animal, is particularly beneficial. Such animals are especially useful in laboratories, animal breeding centres and animal houses, where humans come into frequent and regular contact with animals or their allergens, often several times a day. In such environments humans may be exposed to animal allergens, for example through ventilation or contact with materials, without direct contact with the animals. Often in such situations, humans develop allergies to the animals, a large proportion of which is likely to be caused by uMUP secretion. Such allergic reactions could be prevented by using hypoallergenic animals. Hypoallergenic animals could also be kept as pets.

The hypoallergenic animal according to the invention may be used in any procedure during which a human comes into contact with an animal or its allergens, for example, research, teaching, testing, breeding and supply of animals, animal maintenance, etc.

According to a second aspect of the present invention, there is provided a transgenic animal wherein the uMUP cluster is flanked by LoxP sites or other sites capable of recombining to delete the intervening uMUP cluster in response to the presence of cre recombinase or another recombinase.

Methods for producing such a transgenic animal will be clear to those skilled in the art based on the discussion above.

The uMUP cluster flanked by the LoxP sites or other equivalent sites can be deleted by contacting the sites with cre recombinase or another equivalent recombinase. The cre recombinase or equivalent recombinase can be controlled so that it is only expressed in particular tissues of the animal, e.g., in only liver tissue. Preferably the transgenic animal according to the second aspect of the present invention also expresses cre recombinase or an equivalent recombinase. Preferably the recombinase is inducibly expressed or is tissue specifically expressed.

Using such a transgenic animal, it will be possible to knock out the uMUP cluster in a facultative or tissue specific manner using cre recombinase or an equivalent recombinase. For example, a liver specific knockout could be generated. This can be done by placing LoxP sites on either side of the uMUP cluster to be deleted (see Strategy 2 discussed herein), then making a mouse, then crossbreeding this mouse with one that expresses cre recombinase, for example in the liver. This results in liver specific deletion of the uMUP cluster owing to recognition of the two LoxP sites by cre recombinase which is only expressed in the liver. Methods using this approach are well established (Lewandoski et al., Nature Reviews Genetic, 2: 743-755, 2001).

The present invention also provides an embryonic stem cell wherein the uMUP complex is flanked by LoxP sites or equivalent sites capable of recombining to delete the uMUP complex in response to the presence of a recombinase. Preferably the embryonic stem cell comprises a gene encoding the recombinase operably linked to control elements enabling the recombinase gene to be inducibly or tissue specifically expressed. Preferably the embryonic stem cell is a non-human stem cell, more preferably a rodent (e.g. mouse or rat) embryonic stem cell.

The invention will now be described in detail, by way of example only, with reference to the following drawings.

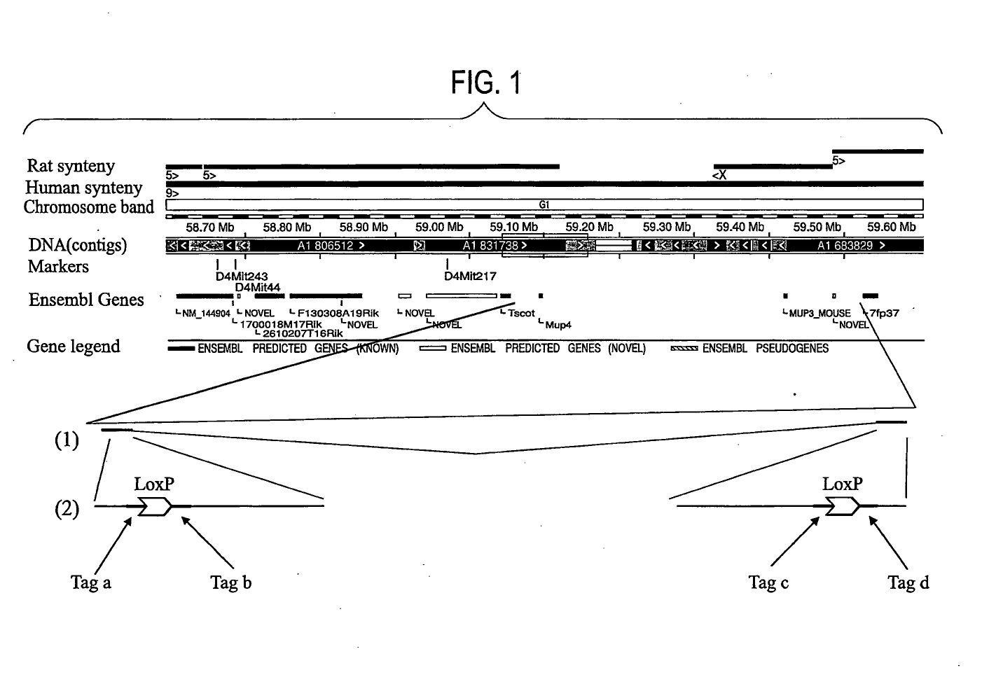

FIG. 1 shows schematically 2 alternative methods of deleting the uMUP complex: method (1) comprises using a single targeting vector that spans the half megabase uMUP region; method (2) comprises using two separate vectors that target each end of the uMUP region and insert LoxP sites in the same orientation, wherein unique short PCR amplifiable tags are inserted at each side of the LoxP sites.

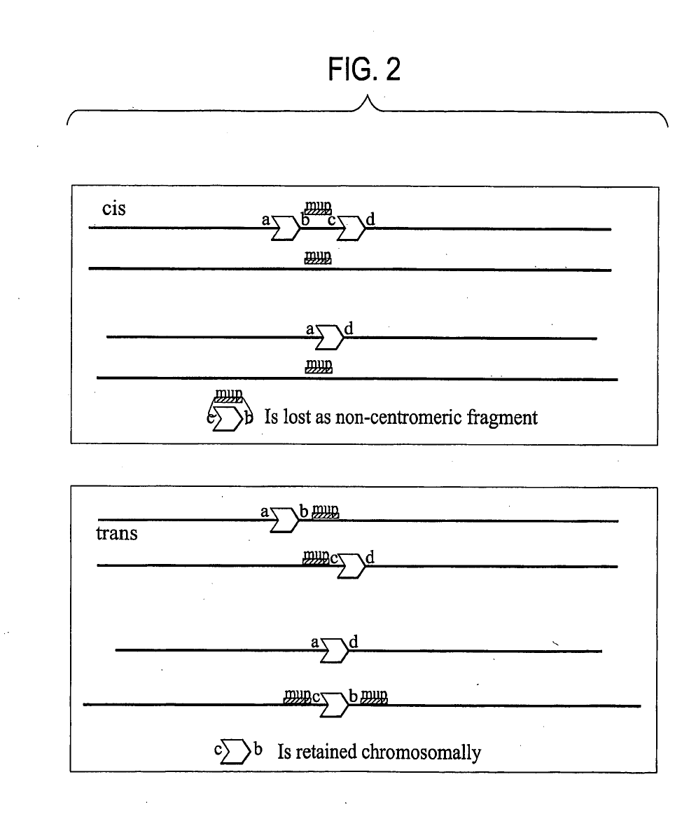

FIG. 2 shows the use of two vectors, and shows the result of the vectors being integrated either cis or trans.

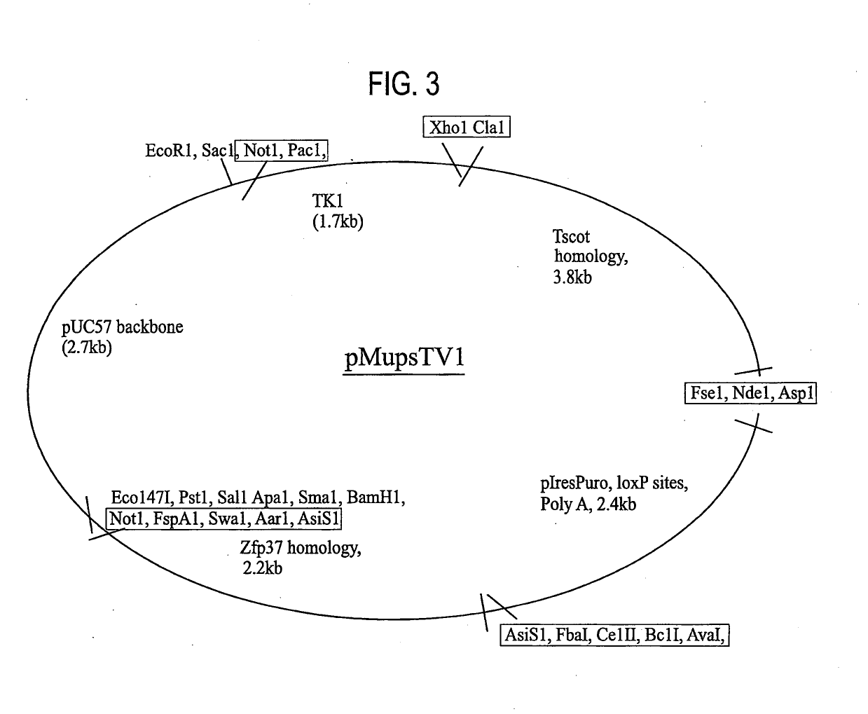

FIG. 3 shows schematically a single vector (pMupsTV1) for deleting the uMUP complex.

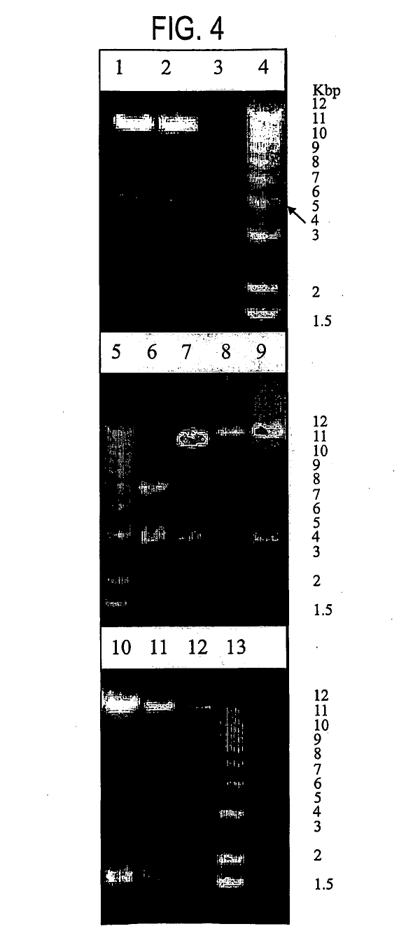

FIG. 4 shows restriction digests demonstrating the correct structure of pMupsTV1. In A lanes 1 and 2 show pMUPSTV1 digested with ClaI and FseI. This excises the Tscot homology arm, which is 3.8 kb long. The brighter band towards the top of the gel is the rest of the vector (9 kb approx). Lane 3 is blank, lane 4 is the 1Kb+DNA size ladder (Invitrogen). The arrow highlights the 4 kb band. In B lane 5 contains the 1Kb+DNA size ladder (Invitrogen). All plasmids shown in lanes 6-9 contain 2 NotI sites, which excise the insert from the pUC57 plasmid backbone. Lane 6 contains the original pMups plasmid obtained from Genscript. Note 2 bands, the pUC57 band (2.7 kb) and a 4.7 kb band that contains the ZFP37 homology, the selection cassette and assorted linkers containing various restriction sites. Lane 7 contains pMUPSTcf6c, which also includes the Tscot arm (3.8 kb). Therefore, the band excised by NotI is now 8.5 kb. Lanes 8 and 9 contain pMUPSTV1. The insert size is now 10.1 kb, following the addition of the TK1 cassette (see FIG. 2). In C lanes 10, 11 and 12 contain pMUPSTV1 cut with PacI and XhoI. This excises the TK1 cassette (approx 1.7 kb). The upper band represents the rest of the construct (11.1 kb). Lane 13 contains the 1Kb+DNA size ladder (Invitrogen). Note: These figures were obtained from a 0.75% agarose gel. Resolution is accurate between 0.5 and 6-7 kb. Bands above this size may migrate higher than they should, especially if more concentrated than the DNA ladder.

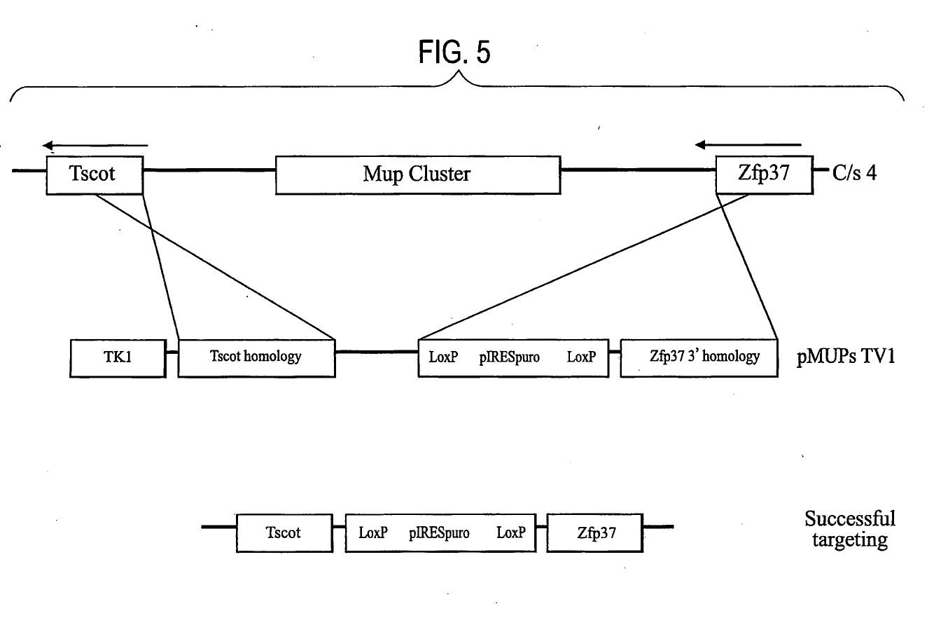

FIG. 5 shows schematically a successful targeting event between the region of mouse chromosome 4 and pMUPs TV 1. A successful targeting results in the loss of the uMUP cluster. As the positive selection cassette is flanked by LoxP sites, an option remains to remove this using cre recombinase at a later date. This could be useful in order to eliminate the possibility that the selection cassette contributes to any phenotype resulting from the targeting. Arrows depict the direction of transcription of the two genes flanking the uMUP cluster. Successful integration of the targeting vector should not disrupt the expression of these genes in any way.

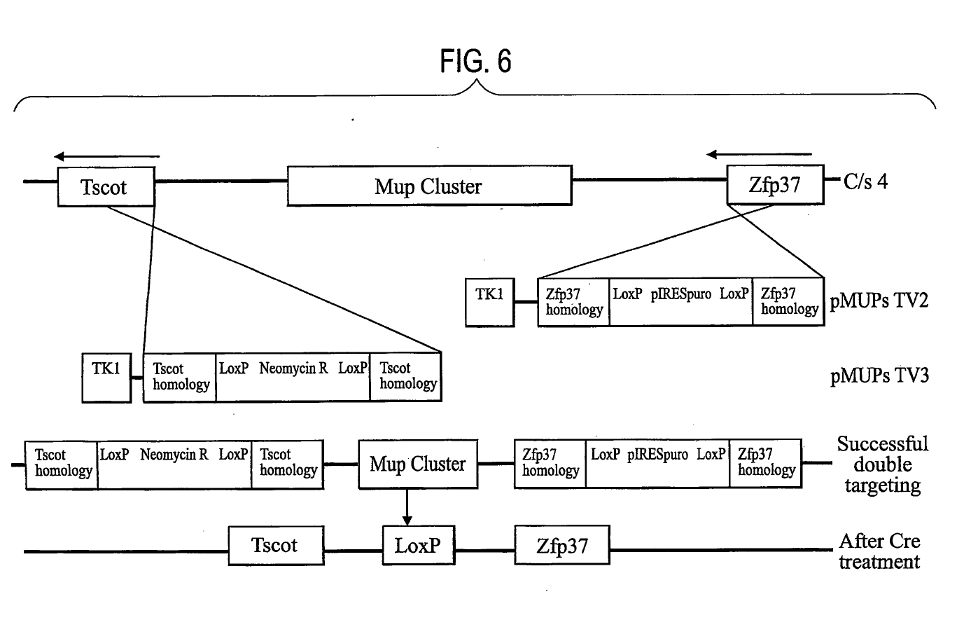

FIG. 6 shows schematically the outcome of successful targeting using two vectors, followed by Cre mediated recombination between LoxP sites, resulting in the loss of the uMUP cluster. Recombination between any of the 4 LoxP sites is possible, selection for successful loss of the uMUP cluster may be accomplished via the addition of 5′ and 3′ segments of an HPRT minigene between the outermost LoxP sites and the relevant ZFP37 and Tscot homologies. Successful recombination between the two outermost LoxP sites results in selectable expression of Hprt, if the ES cell line used for targeting is Hprt negative (e.g., HM1).

### EXAMPLES

### MUP Knockouts as a Research Tool

To establish whether uMUPs and their bound ligands are essential involatile components for the recognition of the sex and identity of conspecifics or their scent marks, and whether uMUPs are the proteins responsible for the binding and release of MHC-associated odours, requires the ability to manipulate the presence or absence of uMUPs and their bound ligands at source. The invention relates to the creation of transgenic animals, especially mice that do not express uMUPs by gene knockout (referred to as uMUP^{-/-}). Unlike other genes such as those comprising the MHC, eliminating the expression of uMUPs is unlikely to have functional consequences beyond scent communication. The ability to eliminate uMUP expression allows the inventors to test the importance of learnt associations between uMUP-ligand complexes and volatiles alone. Normal wild-type mice inevitably will be exposed to their own and maternal uMUPs during rearing but, by eliminating uMUP expression genetically, the inventors are able to control the exposure of mice to uMUPs completely, even from birth. The inventors are thus able to address both the fundamental molecular basis of sex and identity information in scents and how experience influences the way that this information is used.

Specifically eliminating uMUP expression also allows investigation of the functional outcome of the substantial investment in uMUP output among male mice within natural social contexts. To date, research has been limited to examining responses to specific scent cues, while the likely effects on territory defence or mate preference have been surmised. The availability of uMUP^{-/-} males can be applied, for example, to examine the dynamics of territorial scent marking, use of these marks to modulate competitive interactions and the overall outcome in terms of competitive advantage and reproductive success for males that invest in uMUPs. Many uMUPs ligands in males also have VNO-mediated reproductive priming effects on females, accelerating puberty, synchronising oestrus and blocking pre-implantation pregnancy in unfamiliar females. The availability of uMUP^{-/-} mice would allow further investigation of the role of uMUPs in delivering priming signals to the VNO and the importance of learnt associations between volatile and involatile components in reproductive priming.

### MUP Genomic Arrangement

Because of their ready accessibility, sexual dimorphism, endocrine control and polymorphic variation, the expression, inheritance and molecular genetics of the uMUP family has been studied extensively, using the proteins as phenotypic markers. MUPs are expressed in several tissues, including salivary, mammary and lachrymal glands and the liver; the proteins synthesised in the liver are secreted into the bloodstream and filtered by the kidney to be released in urine. The urinary MUP (uMUP) genes in particular have been unambiguously mapped to a tightly linked unit on chromosome 4, mapped by hamster:mouse somatic cell hybrids (Krauter et al. 1982; Bennett et al. 1982; Bishop et al. 1982), recombinant mouse strains (Bennett et al. 1982) and genomic DNA mapping studies (Bishop et al. 1982). These studies suggest that a single complex on chromosome 4 encodes approximately 35 genes (Type 1) and pseudogenes (Type 2).

The current version of the C57BL/6J mouse genome sequence has loci distributed over several chromosomes that share sequence identity (at protein and nucleotide levels) with MUPs. However, detailed examination of these loci confirms that uMUPs are encoded in a cluster on chromosome 4. This is entirely consistent with previous genomic analyses, mapping and linkage studies. Other MUP-like sequences elsewhere in the mouse genome encode MUPs that are expressed in other tissues (e.g. chromosome 7: salivary/mammary glands) or which show weak homology to uMUPs (chromosome 2). The latter sequences contain structural motifs, in particular the N-terminally located GXW triad, which suggests that these genes encode lipocalins, but they are clearly not uMUPs. The chromosome 4 homologies all map to the middle of contig NT_039262 and extend over a 1 Mbp region from 59-60 Mbp, which is in very good agreement with the original proposal of a 720 kb region proposed by Bishop et al. (1982). This region of chromosome 4 is syntenic with a 750 kb region of rat chromosome that has been shown by FISH to encode the equivalent rat αa2u proteins (McFadyen & Locke 2000).

The inventors' analysis of the C57BL/6J genome indicates 15 regions of homology that include the urinary MUP genes 1, 3, 4 and 5; some of the other MUP-like sequences may be pseudogenes. Exhaustive analysis of this region of chromosome 4 has confirmed that there are no genes in this region other than MUPs or pseudogenes. Thus, deletion of the entire 1 Mbp will be effective in ablating every uMUP gene, and carries no risk of deletion of other passenger genes that might interfere with the knockout phenotype.

Creating Transgenic Animals, in Particular Mice, which have the uMUP Complex Deleted.

Several factors influence the choice of inbred strain to use for the initial uMUP knockout. C57BL/6J (B6) has the advantage of full genomic map and sequence availability and is a potentially better described mouse strain for behavioural studies. Conversely 129Sv/Ev is the best available for gene knockout studies with numbers of well proven and characterised ES cell lines. The full genomic sequence for the 129 strain uMUP region of chromosome 4 is not, however, available.

The chromosome 4 uMUP region is flanked by Tscot—a putative thymic stromal co-transporter gene mapping between nucleotides 59173225 and 59183155 and the zinc-finger gene Zfp37 mapping between 60517046 and 60535864 bp. The first uMUP gene is MUP4 at 59224905-59228764 thus leaving an intergenic region of about 41 kb. The last locus in this region with a sequence homology to the MUPs is the ensemble predicted novel transcript ENSMUSG00000044500 mapping at 60410982-60414792 bp which therefore leaves an intergenic region before Zfp37 of 38 kb. Distance estimates vary according to database; however, searches of the NCBI database give the following results. A NCBI mouse genome BLAST search using MUP2 nucleotide sequence reveals that matches to mouse chromosome 4 yield 13 annotated transcripts (MUP4, LOC209155, LOC384019, LOC384020, LOC384021, LOC384022, MUP1, LOC384023/MUP5, LOC384024, LOC384025, LOC236060, LOC381530, LOC381531/MUP3), there are also additional regions of MUP homology (probably pseudogenes). MUP homologies are found in an approximately 1 Mbp region (1253439 bp) from 59224905-60478344bp (MUP4 and LOC381531/MUP3 represent the 5′ most and 3′ most homologies), using the numbering system from the contig NT_039262.2 flatfile. MUP4 is found at 59224905-59228764bp and LOC381531 at 60475343-60478344. Using this method of assessment, the flanking genes, Tscot and Zfp37, are positioned at 59173225-59183155bp and 60517046-60535930bp respectively. This leaves a non-coding region of 41750bp between Tscot and MUP4, and a similar gap of 38702bp between LOC381531/MUP3 and Zfp37. Differences in uMUP cluster distance estimates between Ensemb1 and NCBI probably reflect difficulties in contig assembly where there are so many MUP homologous DNA sequences within a relatively short region of DNA. This should not affect the removal of the uMUP complex as targeting vector(s) homologous to regions of DNA outside the repetitions of the chromosome 4 MUP cluster may be used. Regardless of the size of the intervening region, the vectors should ensure that, either via internal ES cell based or by Cre/LoxP mediated recombination, the uMUP cluster is excised.

Standard techniques for the creation of transgenic animals may be used, and these are know to those skilled in the art. For example, an embryonic stem cell from the relevant species, e.g. mouse, which has an intact uMUP complex, is provided. One or more targeting vectors, as detailed in strategies one and two below are provided. The vectors are introduced into the embryonic stem cell under conditions to allow removal of the uMUP complex to create an embryonic stem cell with no uMUP complex. The embryonic stem cell is then introduced into a blastocyst which is implanted into the uterus of a pseudo pregnant female. The blastocyst is allowed to gestate and is later delivered. The resulting animal is then bred with other such animals and selected for homozygosity and heterozygosity of the deleted uMUP complex. The animal is tested for germline transmission of the uMUP deletion via PCR and/or Southern Blotting.

Referring to FIG. 1 there are two strategies for removal of the uMUP complex.

Strategy 1: A single targeting vector with long homology arms embracing the full deletion is used. These long arms increase the homologous recombination frequency, but may make Southern blot analysis difficult. As this is a large deletion, the cell clones which have integrated the vector are first be screened by interphase FISH for the uMUP region. Clones in which there is only a single interphase hybridisation will be further screened by FISH on metaphase chromosome spreads to confirm that only a single chromosome 4 retains the MUP complex. This is the fastest strategy.

In particular, strategy 1 involves the use of a single replacement vector pMupsTV1 (see FIG. 3). The vector comprises a puromycin resistance cassette, associated with an internal ribosome entry site (IRES) that will only work if it integrates downstream of an existing gene promoter and is transcribed as part of an mRNA. The IRES then allows independent translation of the puromycin resistance gene (Puro). This IRESpuro resistance cassette is flanked by two anus bearing homology to the two genes flanking the uMUP cluster on either side. One arm is homologous to the 3′ end of the ZFP37 gene. Correct integration of one side of the vector into this site will ensure transcription and translation of the resistance cassette, generating puromycin resistant cells (see FIG. 5). This selection strategy is known as promoter trapping, further examples of this technique are described elsewhere (Stanford et al., 2001). The other arm is homologous to a region flanking Tscot, the gene on the other side of the uMUP cluster. This arm of the vector is longer (3.8 kb) and is therefore more likely to integrate via homologous recombination. At the end of this arm is a negative selection cassette, the thymidine kinase gene (TK1). Cells which have correctly integrated the vector at the site of Tscot homology will lose the TK1 gene from the end of the vector and will be resistant to gancyclovir. Random integration of the vector should not result in the removal of TK1 from the end of the Tscot homology arm, thereby rendering the cells sensitive to gancyclovir. Under a regime of double selection using both puromycin and gancyclovir in the culture medium, Puro and gancyclovir resistant cells should have integrated the vector arms at the correct sites, thereby deleting the intervening segment containing the uMUP cluster (see FIG. 5). In order to avoid any false positives, screening of embryonic stem (ES) cell DNA via long range PCR, Southern blot and optionally fluorescence in situ hybridization (FISH) is undertaken. Similar positive/negative selection strategies, sometimes incorporating gene/promoter trap elements, have been used to create a large number of knockouts and even some deletions (Joyner et al., 2000, Stanford et al., 2001, Ciavatta et al., 1995). 50% of pMupsTV1 vector DNA was synthesized by Genescript and sequence data supplied confirming its construction as designed. This sequence was cloned into pUC57, which supplies the replicative plasmid backbone. The 3.8 kb Tscot homology arm was amplified using a long range PCR strategy (Invitrogen, Accuprime Pfx DNA polymerase), incorporating ClaI and FseI sites into the PCR primers, and cloned into the Genscript plasmid (pMups) asymmetrically using the ClaI and FseI sites. The negative selection cassette (the thymidine kinase gene, TK1) was then amplified from a previously successful vector (Mansergh et al., 2005) using a similar PCR strategy incorporating PacI and XhoI sites into the PCR primers. These sites were then used to incorporate TK1 into this vector. Restriction digestion of the finished vector has been used to confirm incorporation of the correctly sized fragments (see FIG. 4). Sequence data for the design of the synthesized portion of the plasmid was supplied by us to Genscript. Sequence of the Zfp37 homology arm was obtained from NCBI, while sequence of the IRESpuro cassette was based on that purchased from Clontech with the plasmid pIRESpuro3, but modified to include 2 LoxP sites. These were included such that on successful integration and excision of the uMUP cluster, the selection cassette can be removed using Cre recombinase. Selection cassettes can sometimes contribute to the phenotypes of mouse models if positioned in a region of DNA which has subsequently proven to be of functional importance.

Strategy 2: Two targeting vectors with different selection cassettes are used, one to integrate at each end of the complex and both bearing a LoxP site. Similar strategies have been used to induce translocations and/or create large scale deletions previously (Ciavatta et al. 1995, Mills and Bradley, 2001, Smith et al. 1995, 2002). Long and shorter arms of homology will allow conventional PCR and Southern blot screening and verification of correct integration. The targeting will be carried out serially. Successfully double-targeted clones may have the two vectors integrated either in cis or trans but because the full contiguous sequence is known they will be in the same orientation. Cre mediated recombination between the two LoxP sites will lead to deletion of the uMUP complex on one chromosome if they are in cis and the other chromosome will be unaltered. If, however, they are in trans the result of recombination will be a chromosomal translocation with deletion on one product and reduplication on the other. The two may be readily experimentally distinguished by including PCR-able tag sequences in the targeting constructs. Similarly a tag sequence on the distal side of the LoxP site may be left on the deleted chromosome in order to facilitate genotyping when breeding the mice (see FIG. 2).

The vector on the ZFP37 side of the uMUP cluster may be based on pMUPSTV1 above. The 3.8 kb Tscot homology arm will be replaced by 2-3 kb homology to further Zfp37 sequence. The vector will then integrate on that side of the uMUP cluster. It contains LoxP sites and will engineer puromycin resistance into correctly targeted ES cells. A further LoxP containing vector may then be designed to integrate on the Tscot side, and may comprise a neomycin resistance cassette. A method for performing such a strategy is shown in FIG. 6.

### Generation of Mup +/− and Mup −/− ES Cells

Mup +/− ES cells will be generated via standard methodology.

A uMUP cluster knockout can be generated from heterozygous ES cells following crossbreeding of heterozygous Fl progeny. The rationale for the generation of homozygous ES cells is that a large market for uMUP null animals in terms of standard research usage. One of the current reasons for the worldwide increase in use of laboratory mice is the generation of thousands of genetically engineered mouse (GEM) models by investigators who are using these to unravel the functions of the genome. Rather than using time consuming breeding methods to generate uMUP null GEM mice, it would be easier for investigators to engineer their models using pre-isolated uMUP null (−/−) ES cells.

Embryos can be isolated from uMUP null mice and null ES cell lines established via standard methods (Joyner et al., 2000).

Generation of homozygous ES cells is also possible by the use of high resistance selection, i.e. administration of high doses of puromycin, G418, or whatever other drug the positive selection cassette engineers resistance to. This results in selection for ES cells which have duplicated the resistance cassette (and therefore the engineered mutation).

Hypoallergenic Mice

The creation of transgenic animals that express no uMUPs could have another, much more general application to research using laboratory mice. Lipocalins are the main cause of laboratory animal allergy (Virtanen et al. 1999), a very common health problem among those working with laboratory animals such as mice and rats. Approximately one-third of exposed people develop symptoms, and about 10% develop asthma (reviewed by Gordon & Preece 2003). Exposure to laboratory animals is one of the top three causes of occupational asthma in the UK (Bush 2001). Urinary MUPs, or equivalent urinary proteins termed á2u globulins in rats, are by far the most prevalent allergens (Schumaker 1980; Wood 2001). Urine samples from 28 different strains of mice from the Jackson Laboratory reveal mean excretion levels of urinary protein of 3.2+0.3 mg/ml among females (n=140) and 13.0±0.7 mg/ml among males (n=140) (unpublished data). With a daily urine output of approximately 1 ml per mouse, the allergen load can be extremely high, particularly when soiled bedding is disturbed.

The risk of sensitisation increases with increasing exposure (Renström et al. 2001; Gordon & Preece 2003) and those working with >50% male rodents experience a four fold increase in risk (Renström et al. 2001), equivalent to the difference in uMUP output between the sexes. Currently, control of exposure involves environmental engineering (e.g. ventilation systems, individually ventilated cage racks), procedural controls and the use of personal protective clothing and respirators (Harrison 2001), but these can be expensive, often uncomfortable or inconvenient to staff and rely on appropriate implementation. High ventilation rates and the distancing of care staff from contact with animals may also compromise animal welfare (Jennings et al. 1998; Baumans et al. 2002).

Eliminating uMUP production would considerably reduce the production of allergens by laboratory mice at source, reducing the risk of sensitisation for the majority of workers and reducing the level of environmental controls required to prevent laboratory animal allergy. However, this would eliminate an important component of the mouse communication system. Before developing such strains, it is essential to confirm that eliminating uMUPs would neither significantly impact the welfare of animals kept under laboratory conditions nor compromise production traits.

In fact, loss of uMUP-mediated communication may not cause significant problems under laboratory conditions and, indeed, might help to reduce problems associated with aggression. Inbred laboratory mice are genetically identical and already unable to discriminate individuals using genetically-determined scents (Boyse et al. 1987; Nevison et al. 2000). uMUPs may be important for recognition between the sexes in breeding stock, but these are externally voided cues that could be provided artificially for the brief period required, using urine or soiled bedding from a strain with normal uMUP expression. No abnormalities were reported in an inbred strain with a spontaneous mutation (now lost) causing very low expression of uMUPs (Duncan et al. 1988).

One of the main functions of uMUPs is in competitive signalling among males. Competitive aggression and territorial defence can cause considerable welfare problems in small cages, resulting in social stress, wounding and sometimes fatal injuries among males of outbred strains or of relatively aggressive inbred strains such as BALB/c (Jennings et al. 1998). This is also likely to increase variability among experimental subjects, but the alternative of single housing induces isolation stress and can be prohibitively expensive. Since uMUPs provide the basis of territorial scent marks and bind volatile male pheromones that stimulate aggression, eliminating uMUP production could reduce the signals that promote aggression between males.

Breeding Homozygous uMUP^{-/-} on Inbred Genetic Backgrounds

The consequences of uMUP knockout within inbred strains can be assessed to provide an initial indication of the overall effects of uMUP^{-/-} on normal behavioural and reproductive functioning for later studies of uMUP function.

Heterozygous F1 uMUP^{+/-} mice on B6 and 129/SvEv genetic backgrounds may be crossed within strain to generate F2 and F3 uMUP^{+/+}, uMUP^{-/-} and uMUP^{+/-} offspring on each inbred background. Weaned offspring may be genotyped. Initial assessment of numbers of each genotype and sex at weaning, weaning weights and home cage behaviour can be used to check for any reduction in viability and development or altered behaviour among uMUP^{-/-} offspring. However, in heterozygous litters, phenotypic differences may be accentuated by differences in behaviour or growth between offspring according to genotype or by genetic similarity between offspring and dam. Offspring from heterozygous pairings will also experience uMUPs produced by mothers and by littermates. Homozygous F2 and F3 from heterozygous litters may be selected and inbred to produce F3 and F4 litters of homozygous uMUP^{-/-} or uMUP^{+/+} controls, resulting in four separate genetic lines varying in background (B6 or 129/SvEv) and uMUP production. In homozygous lines, offspring will only have experience of their own homozygous genotype. Additional F2 and F3 heterozygous uMUP^{+/-} offspring may be crossed with wild mice to examine the functional significance of uMUPs against a normal heterogeneous genetic background (see below).

Molecular Phenotyping of uMUP Knockouts

Urine samples may be obtained from F2 offspring of both sexes post-weaning (uMUP^{+/+}, uMUP^{+/-} and uMUP^{-/-}). Overall expression of uMUPs may be assessed by quantitative western blotting (specific antisera already available). Most (>90%) of 2-sec butyl 4-5 dihydrothiazole (‘thiazole’) and 3-4 dehydro-exo-brevicomin (‘brevicomin’) are uMUP-bound. The absence of uMUPs could therefore lead to these ligands being expressed in normal levels, but not associated with protein. Alternatively, the uMUPs might, en route from the liver, serve as protective vehicles for the pheromones. Under these circumstances, the lack of vehicle might result in the absence of any measurable ligands, even in freshly expressed urine. All volatiles may be assessed using hexane extracted samples, resolved on Carbowax columns using our Polaris/Trace GC/Ion Trap mass spectrometer in selected ion monitoring mode. Full mass scans (m/z 100-500) will also screen for changes in other volatile constituents.

If free ligands are present in urine, they will be lost from deposited urine scent marks at rates much higher than their protein bound counterparts. There is a standardised procedure to assess ligand release (Robertson et al. 2001). Urine samples (10 μl) are deposited on replicate 1.5 cm dia glass fibre disks that are suspended and open to air. At different times after deposition, a disk is removed, ligands are extracted and analysed by GC/MS. The rate of release of thiazole and brevicomin is monotonic and can usually be explained as a single or double exponential (depending on whether ligands are protein bound).

In the event that any uMUPS remain in the urine, further minor uMUP associated regions may be removed by standard techniques.

### Behavioural, Developmental and Physiological Consequences

In addition to using uMUP knockouts to address the functional significance of uMUPs in scent communication, the developmental, reproductive and behavioural consequences of eliminating uMUP production within inbred strains of laboratory mice may be assessed in order to obtain information as to the effect of eliminating uMUP expression on welfare and production under laboratory housing conditions

The following tests all use standard procedures known to those skilled in the art.

Reproductive success may be compared between F4 homozygous uMUP^{-/-} and control litters (n=10 pairs of each for two successive litters), including time from F3 pairing to first and second litters, percent successful pairings, number of offspring born, sex ratio, number of offspring weaned, weight at birth and weaning, percent survival. Pup feeding and maternal behaviour may also be examined to check that there is no abnormality in mother-pup interactions (% time spent suckling, pup retrieval and pup grooming). If reduced success in breeding is found, the effects of providing male and/or female scent cues from uMUP^{+/+} mice will be investigated by (i) painting uMUP^{-/-} mice with urine from normal mice of their own sex, particularly around the anogenital area, (ii) applying urine from normal mice of the opposite sex to the nares of paired uMUP^{-/-} mice, or (iii) housing the mice on substrate soiled by normal mice (from one or both sexes).

Growth rate might increase in mice that are not investing in uMUP production, particularly among post-pubertal males when both growth rate and urinary uMUP production are maximised. Body weight may be measured weekly from weaning until asymptotic adult weight is reached among F3 and F4 mice of both sexes.

Normal behaviour and welfare may be assessed among mice weaned into single sex groups of three of the same genotype by video recording behaviour in the home cage over 24 h (shortly after weaning, once adult and aged 3-4 mo). Activity rhythms; propensity to develop stereotypical behaviour patterns; proportion of time spent chewing at cage bars, which reflects an attempt to escape from the home cage and is considerably increased among animals under poor social or environmental home cage conditions (Hurst et al. 1996; Lewis 2003; Lewis & Hurst 2004); and duration of exploratory behaviour and the type and duration of different social behaviours (Hurst et al. 1996; Nevison et al. 1999) may all be compared.

Aggression may be decreased among uMUP^{-/-} mice if either the production of male pheromones that stimulate aggression, or sensitivity to these scents, is reduced. Adult males of B6 and related strains show relatively low levels of aggression when males have lived together since weaning but this is elevated if males are reintroduced after single housing, are exposed to females or female scents, or home cages are contaminated with scents from other strains. Less is known about aggression within 129/Sv strains, but these are reported to show very similar behaviour to BALB strains with relatively high levels of aggression (Lathe 1996; Le Roy et al. 2000). Differences in aggression within trios of uMUP^{-/-} or control adult males on each genetic background when males are familiar cagemates and when unfamiliar males are introduced after one week of single housing may be compared.

Olfactory sensitivity to urinary scent stimuli. Two factors might reduce the general olfactory sensitivity and interest of mice in social scent stimuli, since the development of neuronal sensory pathways is often dependent on early sensory experience and responses to social stimuli often require prior experience. The extent of investigatory interest when mice encounter (i) urine from uMUP^{-/-} mice of the same strain, (ii) urine from uMUP^{-/-} mice of a different strain, (iii) urine from control mice of the same strain, (iv) urine from control mice of a different strain may be checked. Responses may be measured during 10 min tests in a clean arena. Mice also show a characteristic pattern of investigatory behaviour on meeting an unfamiliar individual, even when these are from the same inbred strain. The duration and type of investigatory behaviour in interactions between different same sex dyads of unfamiliar uMUP^{-/-} and control mice may be examined.

Role of uMUPs in Chemical Communication

To address the function of uMUPs in scent communication, it is possible to compare the action of a wild derived mouse with that of a homozygous uMUP^{-/-} mouse. This avoids many of the pitfalls associated with the use of laboratory mice particularly in studies of mate choice, competitive behaviour and individual recognition where inbred strains often do not exhibit behaviour typical of wild mice. Either wild-derived heterozygous or inbred homozygous genetic backgrounds may be used for stimulus animals and scent donors as appropriate to the questions addressed.

### Female Recognition of Male Scents

To test whether uMUPs and their bound ligands are responsible for the innate recognition of conspecific male scent by females, a comparison may be made between the reaction of a female mouse to the urine of a wild-type or wild-derived male mouse and a uMUP^{-/-} male mouse.

To investigate whether females are able to learn an association between uMUP-ligand complexes and volatiles alone, naive females are exposed repeatedly to full contact with scents from either uMP^{-/-} or control males.

### Male Recognition of Female Scents

To examine whether uMUPs or uMUP-ligand complexes in female urine are responsible for male recognition of female scents wild-derived males are presented with scents from adult males or females that are uMUP^{-/-}, uMUP^{w/w} or uMUP^{+/+}.

Since male recognition of female scents also appears to depend on prior experience (Dizinno et al., 1978), we investigate whether males are able to learn an association between uMUP-ligand complexes and volatiles alone.

### Individual Recognition

Here, we investigate whether females use uMUP patterns to recognise familiar female nesting partners. Wild-derived female subjects are housed from weaning with an unrelated female cagemate. Once highly familiar, we confirm that subject females placed in a clean test enclosure prefer a nest containing scent of the familiar cagemate (or the cagemate behind a barrier) to an equivalent nest of an unfamiliar female, regardless of direct contact with the scent. Females are then given a similar choice between scents from two sisters of the cagemate where one sister expresses the same uMUP type as the cagemate while the other does not.

If females use involatile uMUP type to recognise the cagemate, they will prefer the sister with the familiar uMUP type to the unfamiliar uMUP type when able to contact the scent source, but not in response to volatiles only (which should be equally similar to the sister cagemate). Females are then exposed to contact with scents from both unfamiliar sisters so that they can learn the association between involatile uMUPs and volatile scents. If females recognise the familiar uMUP pattern of their cagemate and update the association with volatiles, they prefer scent from the sister with the familiar uMUP type to the unfamiliar uMUP type even when presented with volatiles only.

To further test the ability of mice to track environmentally-induced changes in the volatile profiles of individuals through a learnt association with uMUPs, we manipulate the volatile scents associated with familiar cagemates by adding artificial odours to their urine scents.

### Molecular Basis of MHC-Associated Odours

To test whether MHC-associated odours result from the differential binding and release of volatiles by fragments of MHC proteins, or are due to MHC-associated differences in volatile metabolites that are bound and released by uMUPs, we test the ability of mice to discriminate between volatiles released from the protein fraction of urine when donors differ only according to MHC (termed H2 in mice) and either express uMUPs or are uMUP^{-/-}.

### Competitive Scent Signalling

The availability of uMUP^{-/-} mice crossed onto a wild-derived background enables us to further address questions concerning the role of uMUPs in competitive scent signalling among male mice. These require relatively simple behavioural experiments and can be carried out making use of the animals according to the invention.

All documents cited herein are hereby incorporated by reference.

### REFERENCES

Bacchini A, Gaetani E, et al. (1992) Pheromone binding proteins of the mouse, Mus musculus. Experientia 48: 419-21.
Baumans V, Schlingmann F, et al. (2002) Individually ventilated cages: beneficial for mice and men? Contemporary Topics in Laboratory Animal Science 41: 13-19.
Beck J A, Lloyd S, Hafezparast M, Lennon-Pierce M, Eppig J T, Festing M F W, Fisher E M C (2000) Genealogies of mouse inbred strains. Nature Genetics 24: 23-25.
Bennett K L, Lalley P A, et al. (1982) Mapping the structural genes coding for the major urinary proteins in the mouse. PNAS 79: 1220-4.
Beynon R J & Hurst J L (2004) Urinary proteins and the modulation of chemical scents in mice and rats. Peptides, 25:1553-1563.
Beynon R J, Hurst J L, et al. (2001). Mice, MUPs and myths: structure-function relationships of the major urinary proteins. Chemical Signals in Vertebrates. A Marchelewska-Koj, D M
Schwarze and J Lepri. New York, Plenum Press. 9: 149-156.
Bishop J O, Clark A J, et al. (1982) Two main groups of mouse major urinary protein genes, both largely located on chromosome 4. Embo J1: 615-20.
Boyse E, Beauchamp G, et al. (1987) The genetics of body scent. Trends Genet 3: 97-102.
Brown R E (1995) What is the role of the immune system in determining individually distinct body odours? Int J Immunopharmacol 17: 655-61.
Brown R E, Singh P B, et al. (1987) The major histocompatibility complex and the chemosensory recognition of individuality in rats. Physiol Behav 40: 65-73.
Bush R (2001) Mechanism and epidemiology of laboratory animal allergy. Ilar J42: 4-11.
Cain K A, Burns T A, et al. (1992) Urinary proteins in four rodent species. Com Biochem Physiol B 101: 199-204.
Carroll L S, Penn D J, et al. (2002) Discrimination of MHC-derived odors by untrained mice is consistent with divergence in peptide-binding region residues. PNAS 99: 2187-92.
Ciavatta D J, Ryan T M, Farmer S C and Townes T M (1995) Mouse model of human beta0 thalassemia: Targeted deletion of the mouse beta maj and beta min-globin gene in embryonic stem cells. PNAS 92 :9259-9263.
Dizinno G, Whitney G, et al. (1978) Ultrasonic vocalizations by male mice in response to a female-produced pheromone: effects of experience. Behav. Biol. 22: 104-113.
D'Udine B & Alleva E (1983). Early experience and sexual preferences in rodents. Mate Choice. P. Bateson. Cambridge, Cambridge University Press: 311-327.
Dulac C & Axel R (1995) A novel family of genes encoding putative pheromone receptors in mammals. Cell 83: 195-206.
Dulac C & Torello AT (2003) Molecular detection of pheromone signals in mammals: from genes to behaviour. Nat Rev Neurosci 4: 551-62.
Duncan R, Matthai R, et al. (1988) Genes that modify expression of major urinary protein in mice. Mol Cell Biol 8: 2705-12.
Egid K & Brown J L (1989) The major histocompatibility complex and female mating preferences in mice. Anim Behav 38: 548-550.
Finlayson J S, Potter M, et al. (1963) Electrophoretic variation and sex dimorphism of the major urinary protein complex in inbred mice: a new genetic marker. J Nat Cancer Inst 31: 91-107.
Gerlai R (1996) Gene-targeting studies of mammalian behavior: is it the mutation or the background genotype? Trends Neurosci 19: 177-81.
Gordon S & Preece R (2003) Prevention of laboratory animal allergy. Occupational Medicine 53: 371-377.
Gosling L M, Roberts S C, et al. (2000) Life history costs of olfactory status signalling in mice. Behav Ecol Sociobiol 48: 328-332.
Guo J, Zhou A, et al. (1997) Urine and urine-derived compounds induce c-fos mRNA expression in accessory olfactory bulb. Neuroreport 8: 1679-83.
Halpern M & Martinez-Marcos A (2003) Structure and function of the vomeronasal system: an update. Prog Neurobiol 70: 245-318.
Harrison D J (2001) Controlling exposure to lab animal allergens. Ilar J42: 17-36.
Held et al., (1987) Mol. Cell. Biol. 7: 3705-3712.
Humphries R E, Robertson D H L, et al. (1999) Unraveling the chemical basis of competitive scent marking in house mice. Anim Behav 58: 1177-1190.
Hurst J L (1993) The priming effects of urine substrate marks on interactions between male house mice, Mus musculus domesticus. Anim Behav 45: 55-81.
Hurst J L, Barnard C J, et al. (1996) Housing and welfare in laboratory rats—Time-budgeting and pathophysiology In single-sex groups. Anim Behav 52: 335-360.
Hurst J L, Beynon R J, et al. (2001). Information in scent signals of competitive social status: the interface between behaviour and chemistry. Chemical Signals in Vertebrates. A.
Hurst J L, Payne C E, et al. (2001) Individual recognition in mice mediated by major urinary proteins. Nature 414: 631-4.
Hurst J L, Thom M D, Nevison C M, Humphries R E & Beynon R J (2004) The “scents” of ownership. Chemical Signals in Vertebrates X (Ed R T Mason & D M Schwarze), (2005)
Hurst J L, Thom M D, Nevison C M, Humphries R E & Beynon R J (2005) MHC odours are not required or sufficient for recognition of individual scent owners. Proceedings of the Royal Society series B, in press.
Hurst J L, Robertson D H L, et al. (1998) Proteins in urine scent marks of male house mice extend the longevity of olfactory signals. Anim Behav 55: 1289-97.
Ivanyi P (1978) Some aspects of the H-2 system, the major histocompatibility system of the mouse. Proc. R. Soc. Lond. B 202: 117-158
Jacob S, McClintock M K, et al. (2002) Paternally inherited HLA alleles are associated with women's choice of male odor. Nat Genet 30: 175-9.
Jemiolo B, Harvey S, et al. (1986) Promotion of the Whitten effect in female mice by synthetic analogs of male urinary constituents. PNAS 83: 4576-9.
Jemiolo D, Alberts J, et al. (1985) Behavioural and endocrine responses of female mice to synthetic analogs of volatile compounds in male urine. Anim Behav 33: 1114-1118.
Jennings M, Batchelor G R, et al. (1998) Refining rodent husbandry: the mouse. Laboratory Animals 32: 233-259.
Johnson et al., (1995) J. Mol. Endocrinol. 14: 21-34.
Joyner A. L. (Ed) Gene Targeting, A Practical Approach (Second Edition). Oxford University Press (USA), 2000.
Konig B (1994) Fitness effects of communal rearing in house mice—the role of relatedness versus familiarity. Anim Behav 48: 1449-1457.
Krauter K, Leinwand L, et al. (1982) Structural genes of the mouse major urinary protein are on chromosome 4. J Cell Biol 94: 414-7.
Kruczek M & Marchlewska-Koj A (1985) Androgen-dependent proteins in the urine of bank voles (Clethrionomys glareolus). J Reprod Fertil 75: 189-92.
Lathe R (1996) Mice, gene targeting and behaviour: more than just genetic background. Trends Neurosci 19: 183-6.
Le Roy I, Pothion S, et al. (2000) Loss of aggression, after transfer onto a C57BL/6J background, in mice carrying a targeted disruption of the neuronal nitric oxide synthase gene. Behav Genet 30: 367-73.
LehmanMcKeeman L D & Caudill D (1991) Quantitation of urinary alpha 2u-globulin and albumin by reverse-phase high performance liquid chromatography. J Pharmacol Meth 26: 239-247.
Leinders-Zufall T, Lane A P, et al. (2000) Ultrasensitive pheromone detection by mammalian vomeronasal neurons. Nature 405: 792-6.
Lewandoski M, 2001 Conditional control of gene expression in the mouse. Nature Reviews Genetics, 2: 743-755
Lewis R S & Hurst J L (2004) The assessment of bar chewing as an escape response in laboratory mice. Animal Welfare, 13:19-25.
Luo M, Fee M S, et al. (2003) Encoding pheromonal signals in the accessory olfactory bulb of behaving mice. Science 299: 1196-201.
Manning C J, Wakeland E K, et al. (1992) Communal nesting patterns in mice implicate MHC genes in kin recognition. Nature 360: 581-583.
Mansergh et al., (2005) Hum. Mol. Genet. 14: 3035-3046.
Marchelewska-Koj, D M Schwarze and J Lepri. New York, Plenum Press. 9: 43-52.
McFadyen D A & Locke J (2000) High-resolution FISH mapping of the rat alpha2u-globulin multigene family. Mamm Genome 11: 292-9.
Mills A A and Bradley A, 2001 From mouse to man: generating megabase chromosome rearrangements. Trends in Genetics, 17(6):331-339.
Moncho-Bogani J, Lanuza E, et al. (2002) Attractive properties of sexual pheromones in mice: innate or learned? Physiol Behav 77: 167-76.
Mucignat-Caretta C & Caretta A (1999) Urinary chemical cues affect light avoidance behaviour in male laboratory mice, Mus musculus. Anim Behav 57: 765-769.
Nevison C M, Armstrong S, et al. (2003) The ownership signature in mouse scent marks is involatile. Proc R Soc Lond B 270: 1957-1963.
Nevison C M, Barnard. C J, et al. (2000) The consequences of inbreeding for recognising competitors. Proc R Soc B 267: 687-694.
Nevison C M, Hurst J L, et al. (1999) Strain-specific effects of cage enrichment in male laboratory mice (Mus musculus). Animal Welfare 8: 361-379.
Novotny M, Harvey S, et al. (1985) Synthetic pheromones that promote inter-male aggression in mice. PNAS 82: 2059-61.
Novotny M V, Ma W, et al. (1999) Positive identification of the puberty-accelerating pheromone of the house mouse: the volatile ligands associating with the major urinary protein. Proc R Soc Lond B 266: 2017-22.
Nyby J, Whitney G, et al. (1978) Postpubertal experience establishes signal value of mammalian sex odor. Behav Biol 22: 545-52.
Nyby J & Zakeski D (1980) Elicitation of male mouse ultrasounds: bladder urine and aged urine from females. Physiol Behav 24: 737-40.
Payne C E, Malone N, et al. (2001). Heterogeneity of major urinary proteins in house mice: population and sex differences. Chemical Signals in Vertebrates. A Marchelewska-Koj, D M Schwarze and J. Lepri. New York, Plenum Press. 9: 233-240.
Penn D & Potts W (1998) MHC-disassortative mating preferences reversed by cross-fostering. Proc R Soc Lond B 265: 1299-306.
Penn D & Potts W K (1998) Chemical signals and parasite-mediated sexual selection. Trends in Ecology & Evolution 13: 391-396.
Penn D & Potts W K (1998) Untrained mice discriminate MHC-determined odors. Physiol Behav 64: 235-43.
Potts W K, Manning C J, et al. (1991) Mating patterns in seminatural populations of mice influenced By MHC genotype. Nature 352: 619-621.
Renstrom A, Karlsson A S, et al. (2001) Worling with male rodents may increase risk of allergy to laboratory animals. Allergy 56: 964-70.
Rich T J & Hurst J L (1999) The competing countermarks hypothesis: reliable assessment of competitive ability by potential mates. Anim Behav 58: 1027-1037.
Robertson D H, Cox K A, et al. (1996) Molecular heterogeneity in the Major Urinary Proteins of the house mouse Mus musculus. Biochem J 316 (Pt 1): 265-72.
Robertson D H L, Beynon R J, et al. (1993) Extraction, characterization and binding analysis of two pheromonally active ligands associated with major urinary protein of house mouse (Musmusculus). J Chem Ecol 19: 1405-1416.
Robertson D H L, Marie A D, et al. (2001). Characteristics of ligand binding and release by major urinary proteins. Chemical Signals in Vertebrates. A Marchlewska-Koj, D M Schwarze and J Lepri. New York, Plenum Press: 169-176.
Schumacher M J (1980) Characterization of allergens from urine and pelts of laboratory mice Mol Immunol 17:1087-95.
Shahan et al., (1987). Mol. Cell. Biol., 7: 1947-1954.
Singer A G, Beauchamp G K, et al. (1997) Volatile signals of the major histocompatibility complex in male mouse urine. PNAS 94: 2210-4.
Singer A G, Tsuchiya H, et al. (1993) Chemistry of odortypes in mice—fractionation and bioassay. J Chem Ecol 19: 569-579.
Singh P B (2001) Chemosensation & genetic individuality. Reproduction 121: 529-39.
Singh P B, Brown R E, et al. (1987) MHC antigens in urine as olfactory recognition cues. Nature 327: 161-4.Smith A J, De Sousa M A, Kwabi-Addo B, Heppel-Parton A, Impey H and Rabbitts(1995) A site directed chromosomal translocation induced in embryonic stem cells by Cre-LoxP recombination. Nature Genetics 9(4):376-85.
Smith A J H, Xian J, Richardson M, Johristone K A and Rabbitts P A (2002). Cre-LoxP chromosome engineering of a targeted deletion in the mouse corresponding to the 3p21.3 region of homozygous loss in human tumours. Oncogene 21:4521-4529.
Stanford W L, Cohn J B and Cordes S P. 2001 Gene trap mutagenesis: past, present and beyond. Nature Reviews Genetics, 2: 756-768.
Szoka P R & Paigen K (1978) Regulation of mouse major urinary protein production by the MUP-A gene. Genetics 90: 597-612.
Utsumi et al., (1999) J. Neurobiol. 39: 227-236.
Virtanen T, Zeiler T, et al. (1999) Important animal allergens are lipocalin proteins: why are they allergenic? Int Arch Allergy Immunol 120: 247-58.
Wedekind C & Furi S (1997) Body odour preferences in men and women: do they aim for specific MHC combinations or simply heterozygosity? Proc R Soc Lond B 264: 1471-9.
Whitney G, Alpern M, et al. (1974) Female odors evoke ultrasounds from male mice. Anim Learn Behav 2: 13-8.
Wood R A (2001) Laboratory animal allergens. Ilar J 42: 12-16.
Yamaguchi T, Inamura K, et al. (2000) Increases in Fos-immunoreactivity after exposure to a combination of two male urinary components in the accessory olfactory bulb of the female rat. Brain Res 876: 211-4.
Yamazaki K, Yamaguchi M, et al. (1979) Recognition among mice. Evidence from the use of a Y-maze differentially scented by congenic mice of different major histocompatibility types. J Exp Med 150: 755-60.

SEQ ID No. 1
AGCACGCACCTGAAACACACTCTGGGATTGAGCCCTGACCCTCCAAATTCGGGAGCATCTCCTAGACTCT
GCAGACAGCAAGCCCTGTTCCCTGTCACGCAGGTCACATGGGTCCAGGGGGCACCTGTCCGTGGAGCAGC
CGGCTCTCTGGCTTCCGGGTGAGGACTTGGATTGAGCCTGTGGTGGCCTCAACCCAGGTGGCCGGCTCCC
TCTACGACGCAGGACTACTCCTGGTAGTGAAGGAGTCCTTCAAGTCTGAGGCTGGAGGCTCCTCTAATTA
CAGTGCCAACCAGTCGCTGGTGGAGTATCAGGAAGACCAACAGCAGAAGGCTATCTCCAATTTCAACATC
ATTTACAACCTCGTGCTGGGCCTGACGCCTCTGCTGTCCGCCTATGGACTGGGCTGGCTCAGTGACCGCT
ACCACCGTAAGATCTCTATCTGCACAGCGATGCTGGGCTTCCTGCTGTCCCGCATCGGACTCTTACTCAA
AGTGATGCTGGACTGGCCAGTGGAGGTGATGTACGGAGCAGCAGCGCTCAATGGGCTATGCGGGAGCTTC
TCCGCTTATTGGTCCGGGGTCATGGCGCTGGGATCCCTGGGCTGCTCCGAAGGCCGCCGCTCCGTGCGCC
TCATCCTCATCGATTTAGTCCTGGGCTTGGCTGGGTTCTCTGGGAGCATGGCTTCGGGGCATCTCTTCAA
GCAAATCGTTGGGCACTCTGCGCAAGGCCTCCTGTTAACTGCCTGCAGCGTTGGCTGTGCGGCGTTTGCC
CTTTTCTACAGCCTCTTTGTGCTGAAGGTCCCTGAGTCCAAGCCCAACAAGGTGCACCCCACTGTAGATA
CTGTGTCTGGCATGATGGGCACCTATCGAACCCTGGATCCAGATCAACAAGACAAACAGAATGTACCAAG
GAACCCTCGGACTCCTCGGAAAGGGAAGTCTTCCCAAAGGGAAGTTGTTGCTCTGCTGTTTGTGGGTGCC
ATCATCTATGACCTGGCTGCTGTGGGCACAGTAGACGTCATGGCCCTTTTTGTGCTTAAGGAGCCTCTCC
ATTGGAACCAAGTGCAGCTGGGCTATGGGATGGCTTCTGGGTACATAATCTTCATAACCAGCTTCTTGGG
TGTCTTGGTTTTCTCCCGCTGCTTCCGGGACACCACGATGATCATAATCGGGATGCTTTCCTTTGGGTCC
GGAGCCCTTCTCTTGGCCTTTGTGAAGGAGACATACATGTTCTACATTGCCCGAGCCATCATGCTGTTTG
CGCTTATTCCGATCACAACCATCAGGTCAGCCATGTCCAAACTCATAAAGGACTCTTCCTATGGAAAGAT
TTTCGTCATACTACAGCTGTGCCTAACTCTGACTGGGGTGGTGACATCCACCATATATAACAAGATCTAT
CAGCTTACGCTGGACAAGTTCATTGGCACCTGCTTTGTTCTATCATCTTTTCTCTCCTTCTTGGCCATTG
TCCCAATTGGTGTTGTGGCCTACAAACAAGTCCCGAGGTCACAACAAGGAGAGTGTGCAGAGAAACAGAG
GAGCTGAAAACGCCAGCCATAACCAGGTTCTGCCTGTGAAACCCAAGCTCCAGTCTTCCAATAACCTGCT
TTGGCCTGCAGGTCCCTGGTGGGAGGGAAAATCACTTTCTGGGTGTTGGAGAAGATTTCCTAGTTCTCAG
ATGCCTCTGCCAGGCTGAGGTTGAGGTCTGTGTGTACCAGCTGCTTGCACAGGTGTTCTGGATCCTGGAG
GGCTTCGGGCTCCAGCTAGCCAGCCCCCTTTTCTCATTGCTGTCTAAAAGACGCCAATGGGGCACATGGA
ATCGTGCTAGTTCTATGGAAGACTGTGACTAAAGCCTAAATTTAACCAACCCAAGGGGAATGAACATGAA
ATGGACATTTTGTAAAAACTGACAACTAAAAACAGTTGTATGCATGACAACAGTTTTTAAATTAATGTAA
ACTCTTTAGAAAAAAAAAAAAAAAAAA
SEQ ID No. 2
ATCGAGGACCTGACAAAGCCAGAGGCGCTGGATCGGAGGAGTGCGGACAAAGCCAGGCGTCCGGAGGAGA
TGGCTACATCCGAGCCTGCGGAAAGCGATGCGGAATGGGAGCAGCTGGAACCTGTGCAGAGAGATGTGTA
CAAGGATACGAAGCTAGAGAACTGCAGCAATCCAGCCTCCATGGGAAATCAAGATCCCAAACAAGACATA
GTCTCCGTGTTGGAAGAAGAAGAGCCATCATCGGGAAAGGGGAAAAAAGCCAGCCCAAGTAGTCTGAAAA
AAATAGCAAGGCCCAAGACAGCAGGAACAAGTGCAAAACTCCAACAAGATGATGAGCATAGGGAGGAAAA
GCAGAAGTCCCAAAGCAAACTTACTAAGGAAGTGACACTCAGGAAGAAAAGTTCCAACAGCAAGAAAAGC
AGTGAGTATGGTTTGTTGGAGAACAAAAGTCTCCACTCAAAACACACTCCTTCCGAGAAAAAACTGCTTA
AGTCCAGTTCCCGTGGGAAGAACTCGAATCAGAATTCAGACTCTCTGAAAAAGAAACCTGACACAGCTAA
TGACCACAGGAAATCACTCAOCCATTCTGCATCTGATGTGAACAAAGATGAAATTCCAACTAGAAAGAAA
TGCGACAAGTTACCCAACAATAAGTTGTCTGATAAAGGTGACAAAAACCAAACCAGCAAAAAATGTGAGA
AAGTATGCCGTCATAGTGCATCCCATACCAAGGAAGACAAAATTCAGACCGGGGAGAAACGGAAATCACA
CTGCCGTACTCCATCTAAACCTGAAAAAGCCCCAGGTTCTGGGAAACCTTATGAATGTAACCACTGTGGG
AAGGTCCTCAGCCATAAACAGGGACTCCTTGACCATCAAAGAACTCACACTGGGGAGAAACCATATGAAT
GTAATGAATGTGGGATAGCTTTCAGCCAGAAGTCCCACCTTGTTGTACATCAGAGAACTCACACTGGGGA
AAAACCATACGAGTGTGAACAGTGTGGCAAAGCACACGGACATAAACATGCCCTCACTGACCATCTAAGA
ATCCATACTGGAGAAAAGCCCTACAAATGTAATGAATGTGGCAAAACGTTTAGACACAGCTCAAACCTTA
TGCAACACCTAAGATCTCACACGGGTGAGAAGCCGTATGAATGTAAGGAATGTGGCAAATCCTTTAGATA
TAATTCATCTCTTACTGAACATGTGAGAACACACACAGGTGAAATACCATACGAATGTAACGAATGTGGC
AAAGCTTTCAAGTATGGCTCATCCCTGACTAAACATATGCGGATTCATACAGGGGAGAAACCATTTGAAT
GTAATGAATGTGGGAAAACTTTTAGCAAAAAGTCACACCTAGTTATACATCAAAGAACTCATACAAAGGA
GAAACCTTATAAATGTGATGAGTGTGGGAAAGCCTTTGGACATAGCTCATCTCTTACCTACCATATGAGA
ACTCATACAGGTGACTGCCCCTTTGAATGTAATCAATGTGGTAAGGCCTTTAAACAGATTGAAGGCCTTA
CCCAACACCAGAGAGTTCACACAGGGGAGAAACCCTATGAGTGTGTTGAATGTGGGAAAGCCTTTAGTCA
GAAGTCACACCTCATCGTACACCAGAGAACTCATACAGGGGAGAAACCCTTTGAATGTTATGAGTGTGGA
AAAGCCTTCAATGCAAAATCACAACTTGTTATTCATCAGAGATCCCACACTGGAGAGAAACCCTATGAAT
GTATTGAATGTGGTAAAGCCTTCAAGCAAAATGCCTCTCTTACCAAACATATGAAAATTCACTCAGAAGA
ACAATCTGAGGAAGAAGATTAATGTAGGAAACCTGACAACTGACTGGTTTGGTATTATTTAACCTTAAAG
ATGTTCTCAATTTGATGATGTTAGAATATCTTTTTTTAGGAAATCATTCCTGGTGATACATGAGAGAATT
TGAATATGGATCTTTACATAAGATGGTAATAAAATTTAACCTTGATCCCAAACCTAAAAAAAAAAAAAAA
AAAAAAAAAAAAAAAAAAAAAAAAAAAAAAAAAAAAAAAAAAAAAAAAAAAAAAAAAAAAAAAAAAAAAA
AAA

1. A transgenic animal from whose genome a uMUP complex has been inactivated or deleted, wherein said inactivation or deletion substantially reduces the production of uMUPs.

2. A transgenic animal according to claim 1 wherein the animal is non-human.

3. A transgenic animal according to claim 2 wherein the animal is a non-human mammal.

4. A transgenic animal according to claim 3 wherein the animal is a rodent.

5. A transgenic animal according to claim 4 wherein the animal is a mouse.

6. A transgenic animal according to claim 5 wherein the uMUP complex is an approximately 1 Mbp complex on chromosome 4.

7. A transgenic animal according to claim 6 wherein the uMUP complex is found between nucleotides 59224905 and 60414792bp.

8. A transgenic animal according to claim 4 wherein the animal is a rat.

9. A transgenic animal according to claim 8 wherein the uMUP complex is found at approximately 78.4 Mbp on chromosome 5.

10. A transgenic animal according to claim 1, wherein the uMUP complex is inactivated by being deleted.

11. A transgenic animal according to claim 1, wherein the animal's uMUP production is reduced to 10% or less of a wildtype animal's uMUP production.

12. A transgenic animal according to claim 11 wherein the animal's uMUP production is reduced to 1% or less of a wildtype animal's uMUP production.

13. A transgenic animal according to claim 1, wherein the animal's uMUP production is eliminated.

14. A method of producing a transgenic animal which has substantially reduced uMUP production, comprising these steps of:
a) providing embryonic stem cells from the relevant animal species comprising an intact uMUP complex;
b) providing one or more targeting vectors capable of inactivating the uMUP complex,
c) introducing the one or more vectors into the embryonic stem cells and selecting for cells with an inactivated uMUP complex;
d) introducing an embryonic stem cell having an inactivated uMUP complex into a blastocyst; and
e) implanting the blastocyst into the uterus of a female animal and allowing it to gestate before delivering the resulting offspring.

15. A method according to claim 14, further including the step of breeding the resulting offspring with similar offspring in order to produce stable animals which are homozygous or heterozygous for the inactivated uMUP complex.

16. A method according to claim 14 or claim 151 wherein the one or more vectors delete the uMUP complex.

17. A method according to claim 14, wherein the one or more vector comprise a single targeting vector with homology arms which embrace the complete uMUP complex.

18 A method according to claim 14, wherein the one or more vector comprise two vectors which flank the uMUP complex

19. A method according to claim 18 wherein the two vector comprise LoxP regions which bring about deletion of the region by Cre-mediated recombination.

20. A method according to claim 14, further comprising the step of inactivating any residual uMUP sites which are not part of the uMUP complex.

21. A vector for use in the method of claim 14.

22. A transgenic animal created by the method of claim 14.

23. An animal which has been bred directly or indirectly from an animal according to claim 22.

24. An animal according to claim 23 which has been bred from an animal homozygous for an inactivated uMUP complex.

25. An animal according to claim 23 which has been bred from an animal heterozygous for an inactivated uMUP complex.

26. A method selecting animals produced by the methods of the invention comprising testing the animals for the inactivation of the uMUP complex.

27. The use of a transgenic animal having substantially reduced uMUP production in behavioral studies.

28. The use of a transgenic animal having substantially reduced uMUP production to establish the molecular mechanism underlying MHC associated odours.

29. The use of transgenic animals having substantially reduced uMUP production to identify agents which affect animal behavior.

30. The use according to claim 25, wherein the agents are selected from agents which attract animals, deter animals, stimulate aggression and reduce aggression.

31. The use of animals having substantially reduced uMUP production as hypoallergenic laboratory animals.

32. The use of animals having substantially reduced uMUP production as pets.

33. An embryonic stem cell that is heterozygous or homozygous for an inactivated uMUP complex.

34. A transgenic animal where the uMUP complex is flanked by LoxP sites or equivalent sites capable of recombining to delete the uMUP complex in response to the presence of a recombinase.

35. The transgenic animal according to claim 34, wherein the transgenic animal expresses the recombinase inducibly or in a tissue specific manner.

36. An embryonic stem cell wherein the uMUP complex is flanked by LoxP sites or equivalent sites capable of recombining to delete the uMUP complex in response to the presence of a recombinase.

37. The embryonic stem cell of claim 36, wherein a gene encoding the recombinase is present and is operably linked to control elements enabling the gene to be inducibly or tissue specifically expressed.
